(19) United States
(12) Patent Application Publication
(10) Pub. No.: US 2017/0020781 A1
(43) Pub. Date: Jan. 26, 2017

(54) FACIAL STEAMING DEVICE

(71) Applicant: PROTV DEVELOPMENT, INC., TAIPEI CITY (TW)

(72) Inventors: JACK TANG, NEW TAIPEI CITY (TW); MICHAEL JIANG, TONGZHOU (CN)

(21) Appl. No.: 14/866,064

(22) Filed: Sep. 25, 2015

(30) Foreign Application Priority Data
Jul. 24, 2015 (TW) 104212016

(51) Int. Cl. A61H 33/12

(52) U.S. Cl. CPC A61H 33/12; A61H 2201/0207

(57) ABSTRACT

A facial steaming apparatus includes a base, a water box and a steam spray head. The base has a steamed object receiving cavity, a water box cavity, a plurality of pores at a bottom wall of the steamed object receiving cavity, and a steam generator. The water box is detachably assembled with the base and located in the water box cavity. The steam spray head is detachably assembled with the base and has a steam outlet in communication with the steamed object receiving cavity. By means of the design that steam spray head is detachable and the base has the steamed object receiving cavity to receive a to-be-steamed object, the facial steaming apparatus of the present disclosure can provide the user to steam face by the steam containing component of fruit or vegetable.

### BACKGROUND

1. Technical Field

The present disclosure relates to a field of facial steaming, more particularly to a facial steaming apparatus.

2. Description of Related Art

China Patent No. CN203291200U discloses a cold and hot spraying facial steaming device which includes a device body, a facial steaming cover and a middle base. The facial steaming cover is connected with the device body through the middle base, and a steam regulating valve is connected with an upper part of the middle base in a buckled mode and further connected with the facial steaming cover in a buckled mode. A plastic cover, an atomizer, a water absorption bar, a copper wire ring and a plastic cover water absorption opening are installed in the facial steaming cover. An aluminum pot is further installed in the device body and positioned below the middle base. A temperature controller, a circuit board and a PTC heater connected with a power line are arranged below the aluminum pot. A base is installed on a lower part of the device body.

The aforesaid cold and hot spraying facial steaming device just sprays cold or hot steam for caring facial skin, but not provides steam with different functions to meet different skincare demands. What is need is to improve the cold and hot spraying facial steaming device for satisfying different skincare demands.

### SUMMARY

A primary objective of the present disclosure is to provide a facial steaming apparatus, which produces steam containing components of fruit or vegetable for satisfying different skincare demands.

According to the primary objective, a facial steaming apparatus of the present disclosure comprises a base, a water box and a steam spray head. The base has a steamed object receiving cavity, a water box cavity, a plurality of pores at a bottom wall of the steamed object receiving cavity, and a water outlet at a sidewall of the water box cavity. The base has an adjusting knob at an outer surface thereof and a steam generator which includes a shell member, a heater and a temperature controller. The shell member has an upward opening and a water inlet in communication with the water outlet. Further, the shell member is combined with the bottom wall of the steamed object receiving cavity to form a steam generating chamber between the shell member and the steamed object receiving cavity. The water box is detachably disposed in the water box cavity of the base, and has at least one water box outlet in communication with the water outlet of the base. The steam spray head is detachably assembled with the base and configured to close the steamed object receiving cavity, and has a steam outlet in communication with the steamed object receiving cavity of the base, such that the steam can be sprayed from the steamed object receiving cavity via the steam outlet.

According to the present disclosure, the steam spray head can be separated from the base, and the base has the steamed object receiving cavity therein to receive a to-be-steamed object. The to-be-steamed object can be fruit, vegetable, honey or milk; hence, the facial steaming apparatus of the present disclosure can spray the steam containing components of the fruit, vegetable, honey or milk for various facial skincare effects.

In order to further understand the techniques, means and effects of the present disclosure, the following detailed descriptions and appended drawings are hereby referred, such that, through which, the purposes, features and aspects of the present disclosure can be thoroughly and concretely appreciated; however, the appended drawings are merely provided for reference and illustration, without any intention to be used for limiting the present disclosure.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

FIG. 1 is a perspective view of a first preferred embodiment of the present disclosure.

FIG. 2 is a perspective exploded view of the first preferred embodiment of the present disclosure, illustrating that a steam spray head, a water box and a base are separated.

FIG. 3 is a perspective view of a main body of the first preferred embodiment of the present disclosure.

FIG. 4 is a cross-sectional view of the water box of the first preferred embodiment of the present disclosure.

FIG. 5 is a perspective exploded view of the water box of the first preferred embodiment of the present disclosure.

FIG. 6 is a cross-sectional view of the first preferred embodiment of the present disclosure, illustrating the water box and the base are separated.

FIG. 7 is a cross-sectional assembly view of the first preferred embodiment of the present disclosure.

FIG. 8 is a perspective cross-sectional view of a part of the first preferred embodiment of the present disclosure.

FIG. 9 is a perspective schematic view of the first preferred embodiment of the present disclosure, illustrating fruits to be placed in the steamed object receiving cavity.

FIG. 10 is a cross-sectional view of the first preferred embodiment of the present disclosure, illustrating that the fruits are placed in the steamed object receiving cavity.

FIG. 11 is a cross-sectional view of the first preferred embodiment of the present disclosure, illustrating that a towel is placed into the steamed object receiving cavity.

FIG. 12 is a perspective schematic view of a second preferred embodiment of the present disclosure, illustrating the fruits to be placed in the steamed object receiving cavity and a UV lamp installed at a bottom wall of the steamed object receiving cavity.

### DESCRIPTION OF THE EXEMPLARY EMBODIMENTS

Reference will now be made in detail to the exemplary embodiments of the present disclosure, examples of which are illustrated in the accompanying drawings. Wherever possible, the same reference numbers are used in the drawings and the description to refer to the same or like parts.

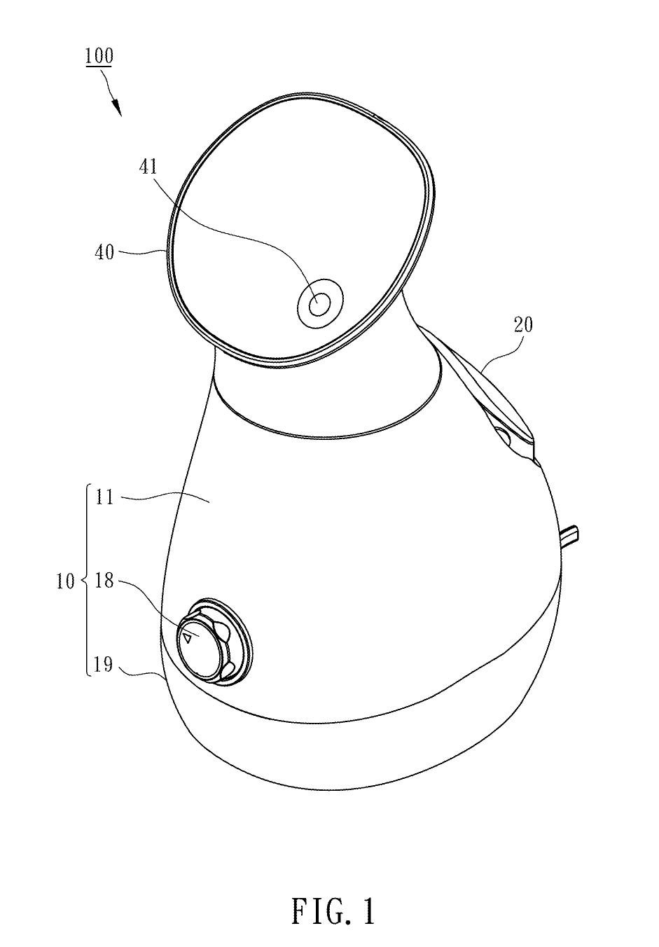

Please refer to FIG. 1 through FIG. 8. A facial steaming apparatus 100 of a first preferred embodiment of the present disclosure comprises a base 10, a water box 20, and a steam spray head 40.

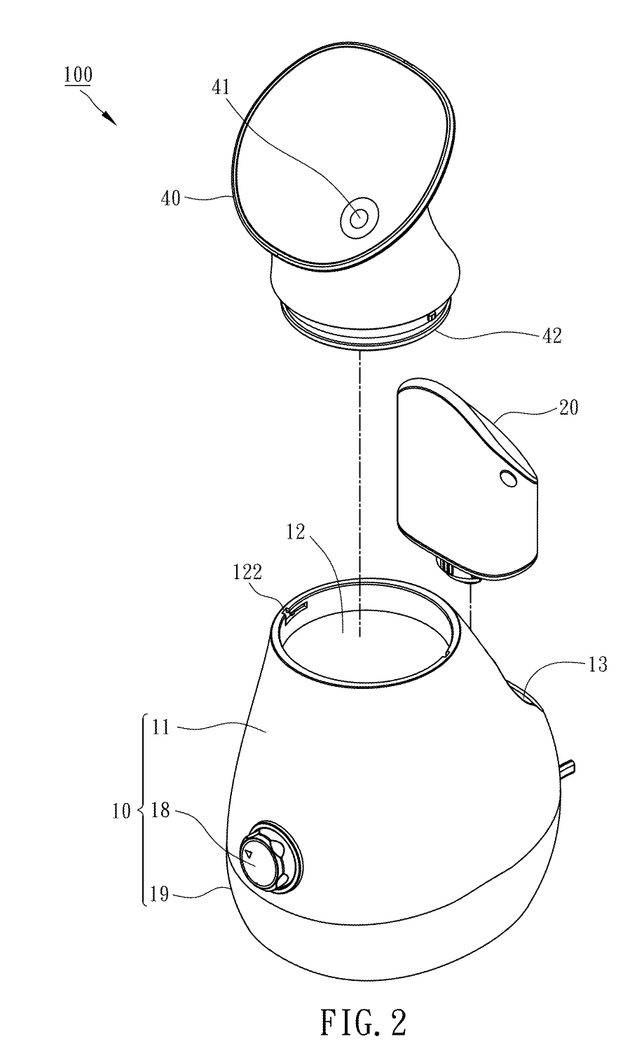

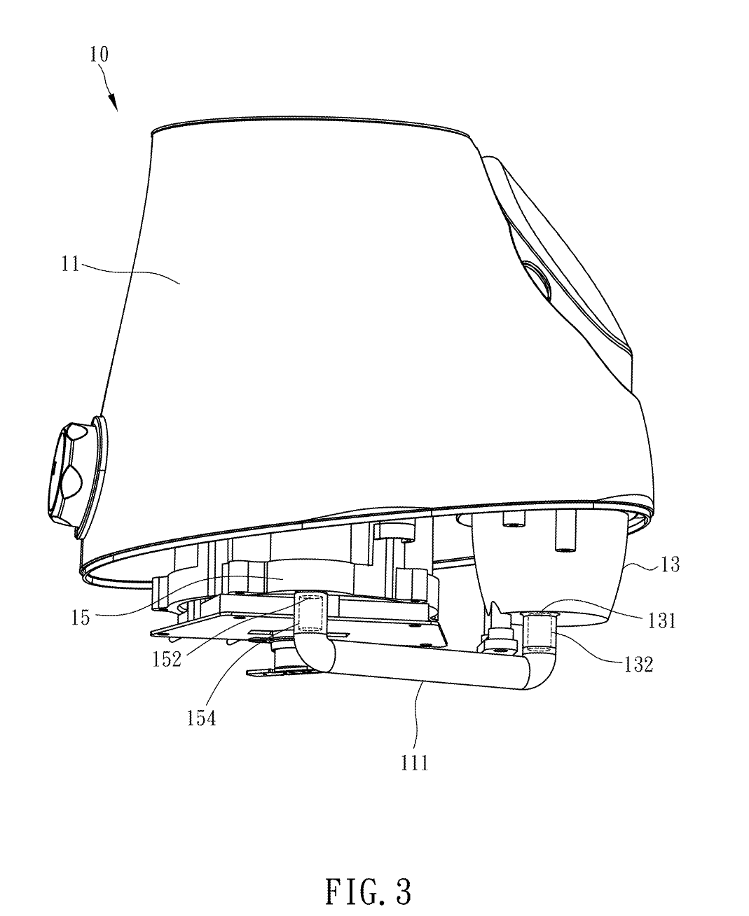

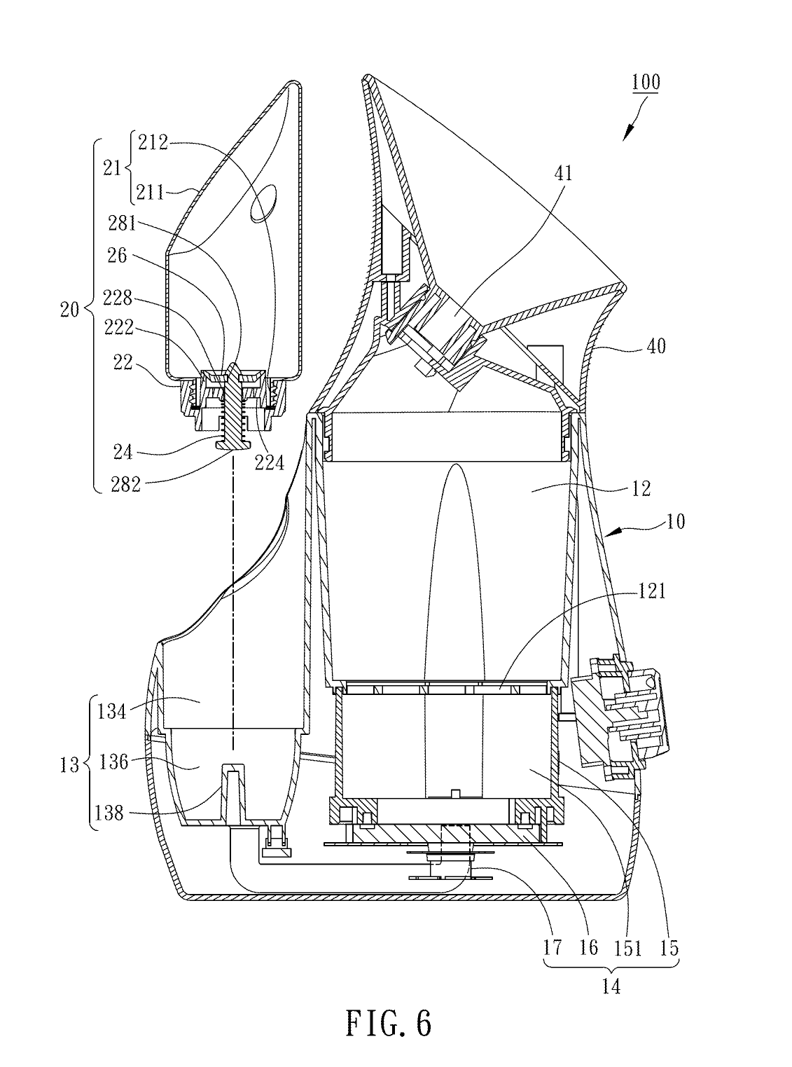

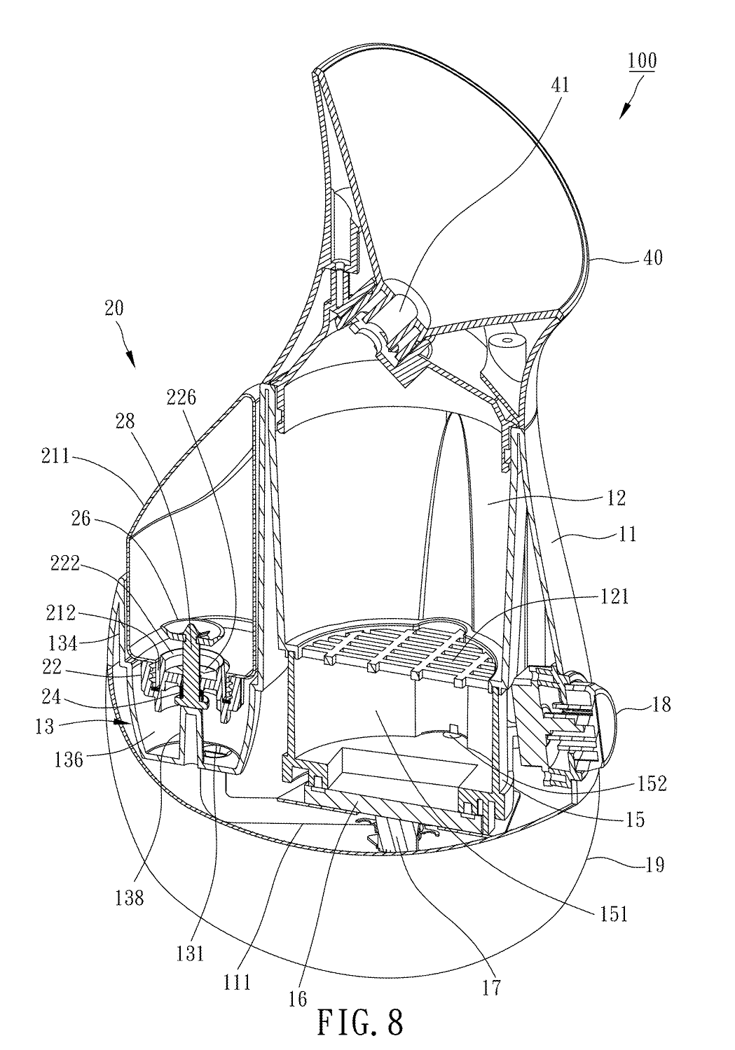

As shown in FIG. 6, the base 10 has a steamed object receiving cavity 12, a water box cavity 13, and a plurality of pores 121 formed on a bottom wall of the steamed object receiving cavity 12. Further, the base 10 has a steam generator 14 including a shell member 15, a heater 16 and a temperature controller 17. The heater 16 and the temperature controller 17 are mounted to the bottom of the shell member 15. The shell member 15 has an upward opening, and is combined with the bottom wall of the steamed object receiving cavity 12 so as to form a steam generating chamber 151 between the shell member 15 and the steamed object receiving cavity 12 As shown in FIG. 8, the shell member 15 has a water inlet 152, and the sidewall of the water box cavity 13 has a water outlet 131 in communication with the water inlet 152, and an adjusting knob 18 is disposed at an outer surface of the base 10. In the first embodiment, as shown in FIGS. 6 and 8, the base 10 has a main body 11 and a bottom cover 19, the steamed object receiving cavity 12 and the water box cavity 13 are located in the main body 11. In addition, as shown in FIG. 3, the base 10 further has a first connection pipe 132 extending from the water outlet 131, a second connection pipe 154 extending from the water inlet 152, and a third connection pipe 111 connected between the first connection pipe 132 and the second connection pipe 154. In addition, as shown in FIG. 6 and FIG. 8, the water box cavity 13 has a first receiving cavity portion 134 and a second receiving cavity portion. The first receiving cavity portion 134 is larger than and located above the second receiving cavity portion 136, and a pillar 138 extends from a bottom wall of the second receiving cavity portion 136 toward the first receiving cavity portion 134, and the water outlet 131 is located at the second receiving cavity portion 136. As shown in FIG. 2, at least one fastening slot 122 is formed on the sidewall of the steamed object receiving cavity 12 of the main body 11, and is two in number as an example in this embodiment. In addition, an adjusting knob 18 is configured to switch a turn-off state, a towel heating state, a fruit-vegetable steaming state and a pure-water steaming state.

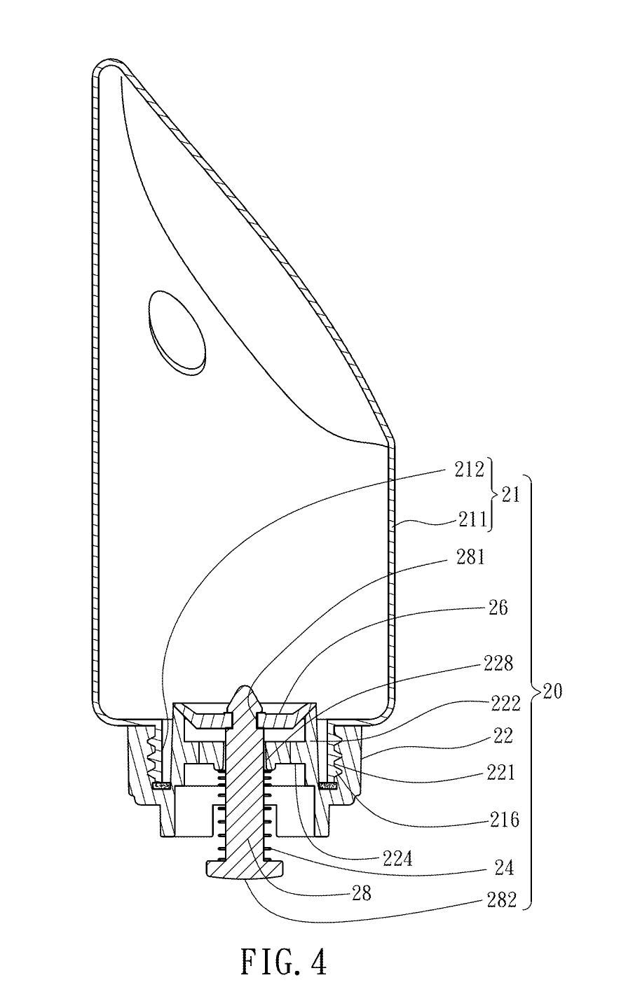

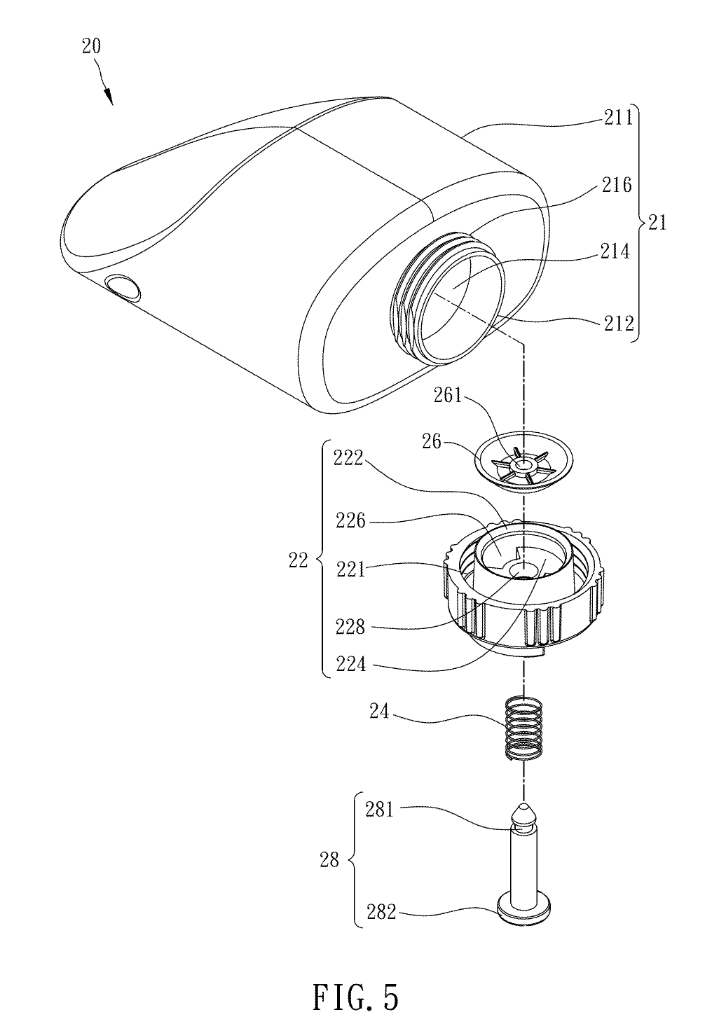

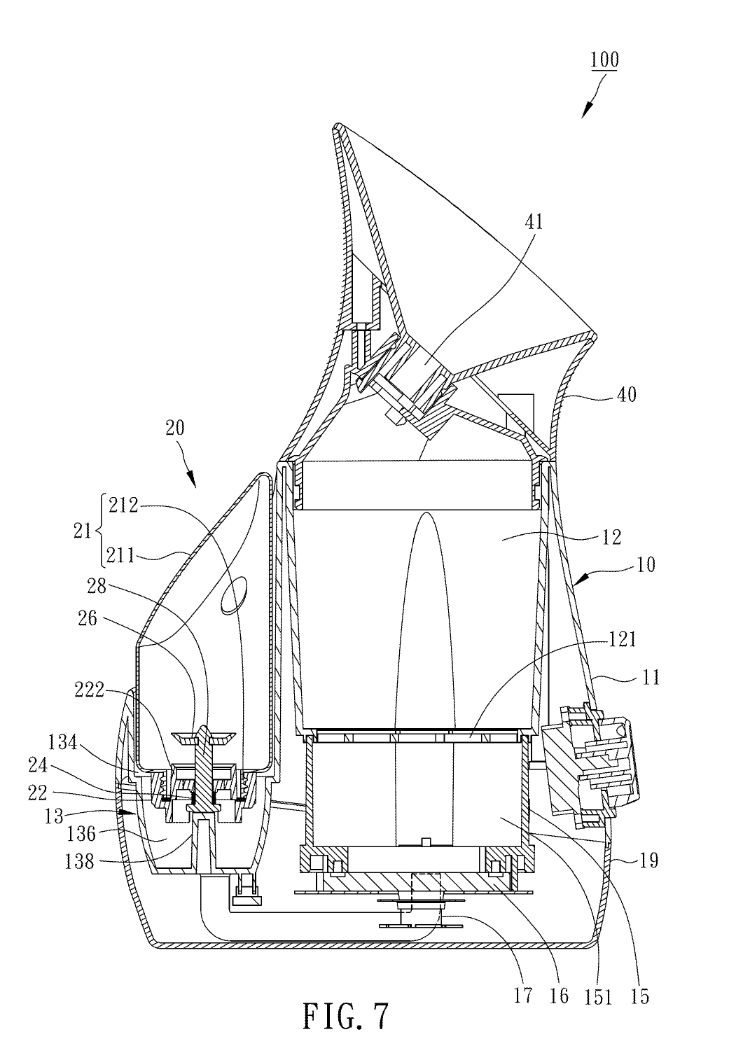

The water box 20 is detachably assembled with the base 10, as shown in FIG. 2, and located in the water box cavity 13. As shown in FIG. 5, the water box 20 has at least one water box outlet 226 which is two in number as an example in this embodiment. As shown in FIG. 8, the two water box outlets 226 are in communication with the water outlet 131 of the base 10. In the first embodiment, as shown in FIGS. 4 and 5, the water box 20 includes a box body 21, a box cover 22, a spring 24, a sealing member 26 and a valve pin 28. The box body 21 includes a body part 211 and a spout 212 which are connected with each other. The spout 212 is formed as a water-filling opening 214, and has an outer thread 216 on an outer surface thereof. The box cover 22 has an inner thread 221 on an inner wall thereof screwed with the outer thread 216 of the spout 212 of the box body 21, a channel 222 extending outwardly from an inner bottom wall thereof and located inside the spout 212 (as shown in FIG. 7) and provided with an outer diameter smaller than an inner diameter of the spout 212, and a division 224 located inside the channel 222 and provided with a through hole 228 at a center thereof, as shown in FIG. 5. The two water box outlets 226 are formed on the division 224, as shown in FIG. 5. The valve pin 28 has an end formed with a mounting slot 281 and other end formed with a constraining block 282, and inserted into the spring 24 and the through hole 228 of the division 224 to constrain the spring 24 between the division 224 and the constraining block 282. As shown in FIG. 4, the sealing member 26 has a fixing hole 261 at a center thereof sleeved on the valve pin 28 and is fastened in the mounting slot 281 of the valve pin 28. When the sealing member 26 is forced to cover the channel 222 subject to the elastic force applied by the spring 24, the channel 222 is closed. Please refer to FIG. 6 and FIG. 7. While the water box 20 is assembled with the main body 11, the body part 211 of the box body 21 is located in the first receiving cavity portion 134 and contacted against the sidewalls of the first receiving cavity portion 134, the spout 212 of the box body 21 and the box cover 22 are located in second receiving cavity portion 136, and the pillar 138 pushes against the valve pin 28 to move the valve pin 28 upwardly to compress the spring 24, so as to make the sealing member 26 leave from the channel 222 and enable the space inside the water box 20 in communication with the second receiving cavity portion 136.

As shown in FIG. 2, the steam spray head 40 has a steam outlet 41, and is detachably assembled with the base 10 and located above the steamed object receiving cavity 12 to close the steamed object receiving cavity 12, so that the steam is sprayed via the steam outlet 41 only. In the first embodiment, the steam spray head 40 has at least one fastening block 42 which is two in number as an example in this embodiment. While the steam spray head 40 is assembled with the base 10, as shown in the FIGS. 1 and 2, the two fastening blocks 42 are respectively fastened with the two fastening slots 122 of the main body 11.

The above description illustrates the structure of the first preferred embodiment of the present disclosure, and the operation of the first preferred embodiment of the present disclosure is illustrated below.

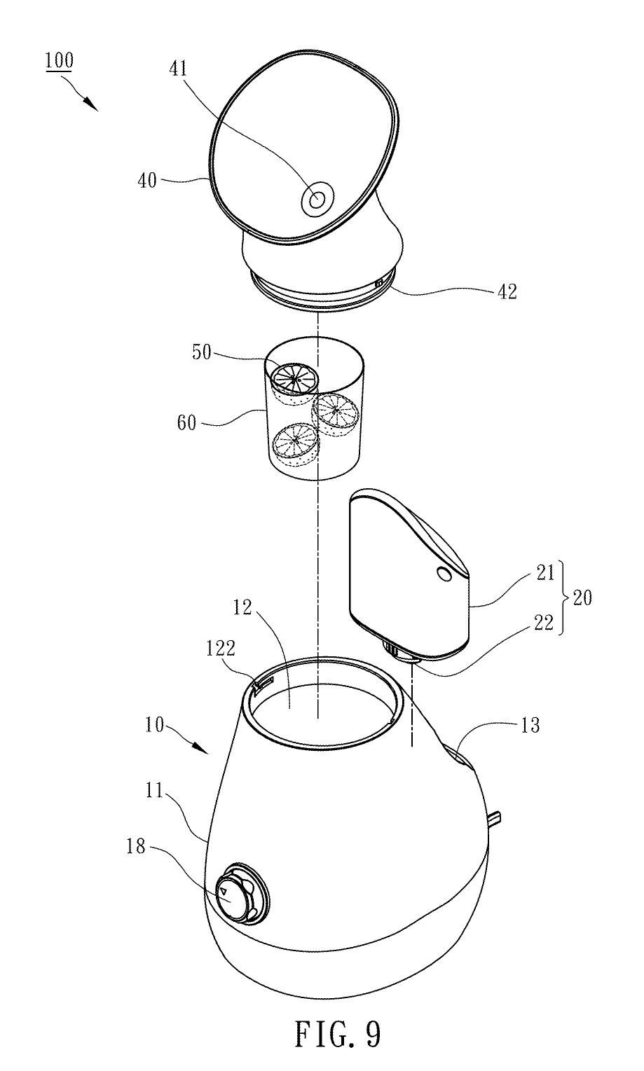

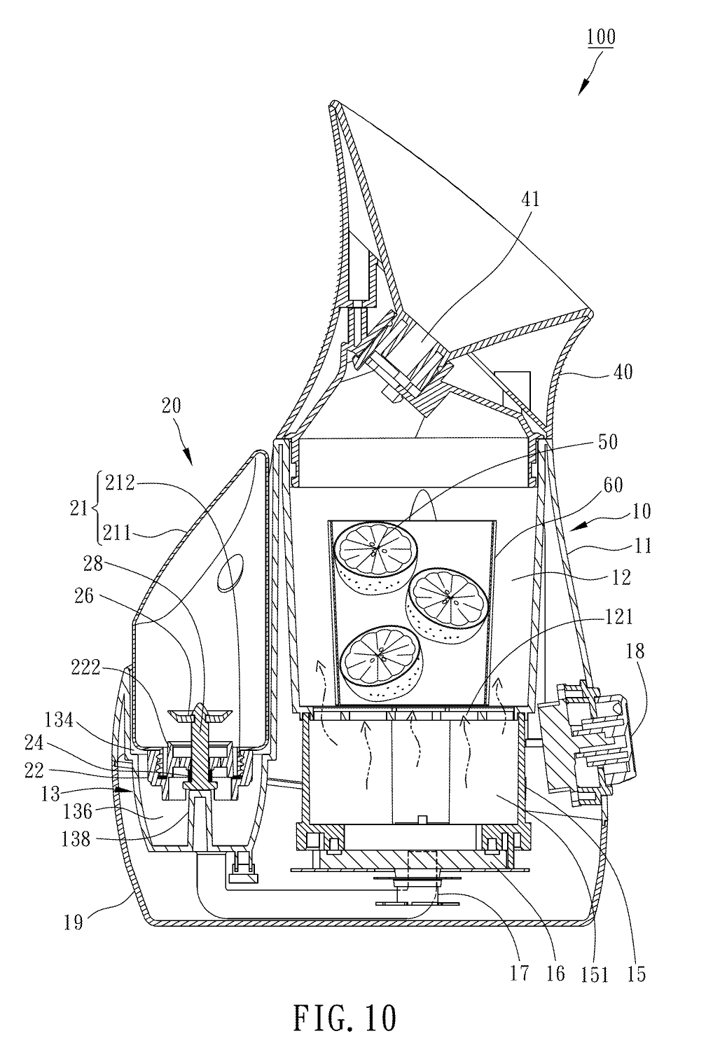

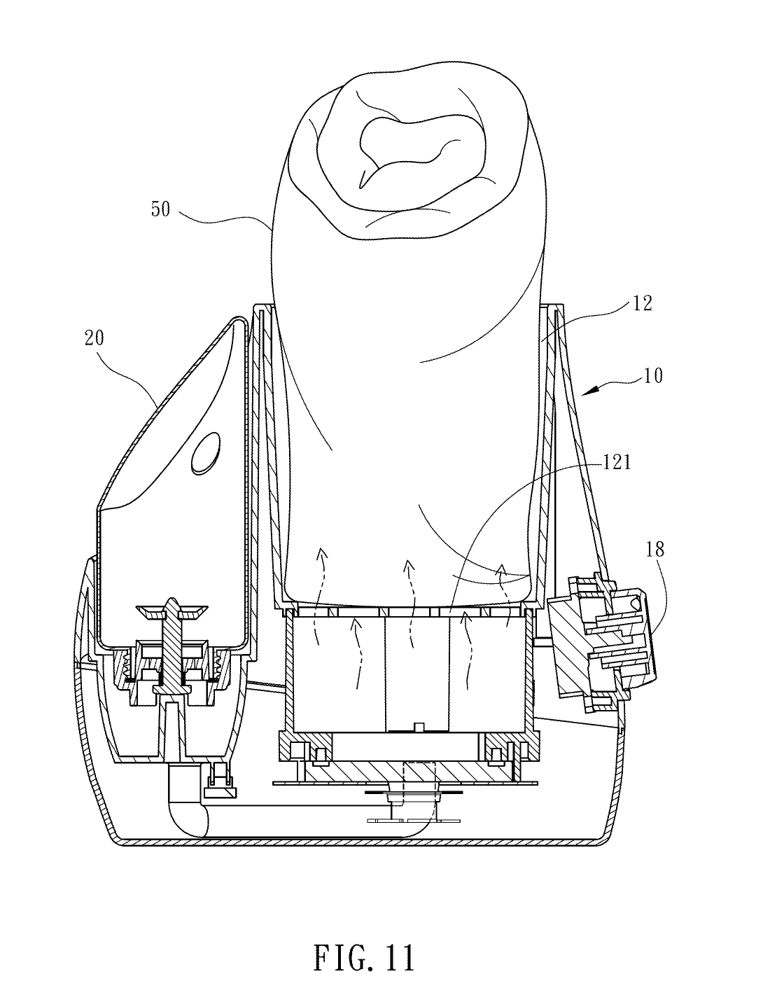

Please refer to FIGS. 9 and 10. When wanting to steam face by the steam containing component of fruit or vegetable, the user can turn the steam spray head 40 to separate the two fastening blocks 42 of the steam spray head 40 from the two fastening slots 122 of the base 10, so as to separate the steam spray head 40 from the base 10. Next, the user can place a to-be-steamed object 50 in a flask 60, such as fruit, vegetable, honey or milk, and then place the flask 60 inside the steamed object receiving cavity 12 of the main body 11. Next, the user fastens the two fastening blocks 42 of the steam spray head 40 with the two fastening slots 122 of the base 10 to assemble the steam spray head 40 with the base 10, and then turn the adjusting knob 18 from the turn-off state to the fruit-vegetable steaming state. Please refer to FIG. 10. When the water box 20 is assembled with the base 10, the body part 211 of the water box 20 is placed into the first receiving cavity portion 134 and abutted against the sidewalls of the first receiving cavity portion 134, the spout 212 of the water box 20 and the box cover 22 are located in the second receiving cavity portion 136, and the pillar 138 pushes against the valve pin 28 to move the valve pin 28 upwardly to compress the spring 24, so as to make sealing member 26 leave from the channel 222. In the meantime, the water contained in the water box 20 flows into the second receiving cavity portion 136 via the two water box outlets 226, as shown in FIG. 8, the water further is flowed into the steam generating chamber 151 via the water outlet 131, the third connection pipe 111, and the water inlet 152 of the shell member 15 and then heated by the heater 16 to product steam. The produced steam enters the steamed object receiving cavity 12 via the pores 121 to heat the to-be-steamed objects 50 in the flask 60. After being steamed, some components of the steamed object 50 are transpired so that the steam contains some components of the steamed objects 50. The steam is then sprayed from the steam outlet 41 of the steam spray head 40, and the user can approach the steam spray head 40 to make his/her face be steamed. Please refer to FIG. 11. When wanting to heat towel, the user can place the towel 50 into the steamed object receiving cavity 12, and then turn the adjusting knob 18 from the turn-off state to the towel heating state, so that the towel can be heated by the steam. The difference between the towel heating state and the fruit-vegetable steaming state is that the object 50 is changed to the towel and the steam spray head 40 is taken off, and other operating steps of the towel heating state are substantially the same as that of the fruit-vegetable steaming state, so their detailed descriptions are omitted. Please refer to FIG. 7. For using pure-water steam to steam face, the user can turn the adjusting knob 18 to the pure-water steaming state, and the main difference between the pure-water streaming state and the fruit-vegetable steaming state is that no to-be-steamed object 50 is placed in the steamed object receiving cavity 12. Other operating steps of the pure-water streaming state are substantially the same as that of the fruit-vegetable steaming state, so their detailed descriptions are omitted.

According to the above description, it is obvious that the present disclosure can solve the problem that the conventional facial steaming apparatus can just spray cold or hot steam for skincare operation. The steam spray head 40 and the base 10 of the present disclosure are detachably assembled with each other, and the base 10 has the steamed object receiving cavity 12 formed therein, so the user can place fruit, vegetable, honey or milk in the steamed object receiving cavity 12 to enable the facial steaming apparatus 100 to spray the steam containing the component of fruit, vegetable, honey or milk for different skincare effects. In addition, the user can place the towel in the facial steaming apparatus 100 for heating.

In above-mentioned description for the first preferred embodiment, the at least one fastening slot 122, the at least one fastening block 42, and the at least one water box outlet 226 are two in number as examples, respectively, but the present disclosure is not limited thereto. In practice, the at least one fastening slot 122, the at least one fastening block 42 and the at least one water box outlet 226 can be one in number, respectively, and their implementation can refer to the first preferred embodiment, so the drawing for illustration is omitted.

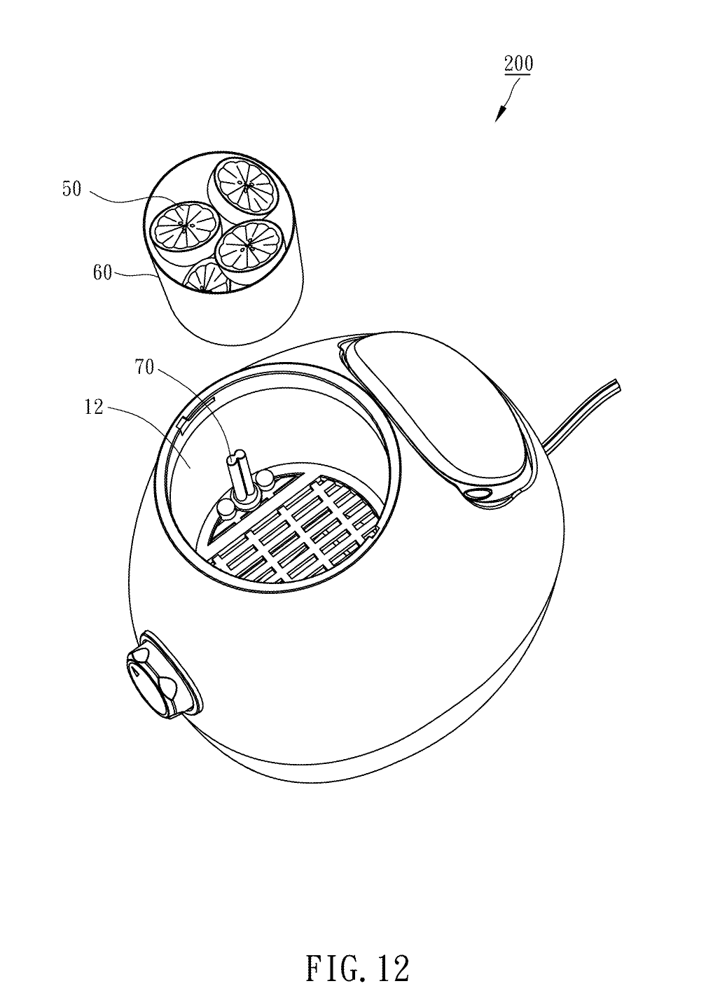

Please refer to FIG. 12 which shows a facial steaming apparatus 200 of a second preferred embodiment of the present disclosure. The different between the second preferred embodiment and the first preferred embodiment is described below.

The facial steaming apparatus 200 further includes a UV lamp 70 disposed in the steamed object receiving cavity 12, and a bottom of the UV lamp 70 is in contact with the bottom wall of the steamed object receiving cavity 12.

As shown in FIG. 12, in using state of the second preferred embodiment of the present disclosure, the UV lamp 70 can emit UV light to sterilize the object 50 placed in the steamed object receiving cavity 12 and the steam containing the component of the steamed object 50.

Other structures of the second preferred embodiment of the present disclosure are substantially the same as that of the first preferred embodiment, so their detailed descriptions are omitted.

The above-mentioned descriptions represent merely the exemplary embodiment of the present disclosure, without any intention to limit the scope of the present disclosure thereto. Various equivalent changes, alternations or modifications based on the claims of present disclosure are all consequently viewed as being embraced by the scope of the present disclosure.

What is claimed is:

1. A facial steaming apparatus, comprising:
a base having a steamed object receiving cavity, a water box cavity, a plurality of pores at a bottom wall of the steamed object receiving cavity, and a water outlet at a sidewall of the water box cavity, the base having an adjusting knob at an outer surface thereof and a steam generator which comprises a shell member mounted to the bottom wall of the steamed object receiving cavity to define a steam generating chamber therebetween and provided with an upward opening and a water inlet in communication with the water outlet, a heater mounted to the shell member and a temperature controller mounted to the shell member;
a water box detachably disposed in the water box cavity of the base, and having at least one water box outlet in communication with the water outlet of the base; and
a steam spray head detachably assembled with the base and configured to close the steamed object receiving cavity and having a steam outlet in communication with the steamed object receiving cavity of the base.

2. The facial steaming apparatus according to claim 1, wherein the base has a main body and a bottom cover, and the steamed object receiving cavity and the water box cavity are disposed in the main body.

3. The facial steaming apparatus according to claim 1, wherein the base further has a first connection pipe extending from the water outlet, a second connection pipe extending from the water inlet, and a third connection pipe connected between the first connection pipe and the second connection pipe.

4. The facial steaming apparatus according to claim 1, wherein the water box cavity has a first receiving cavity portion, a second receiving cavity portion, and a pillar extending from a bottom wall of the second receiving cavity portion toward the first receiving cavity portion; the first receiving cavity portion is larger than and located above the second receiving cavity portion; the water outlet is located at the second receiving cavity portion.

5. The facial steaming apparatus according to claim 4, wherein the water box comprises a box body, a box cover, a spring, a sealing member and a valve pin;
wherein the box body comprises a body part and a spout connected with the body part and formed with a water-filling opening and has an outer thread formed at an outer surface thereof;
wherein the box cover has an inner thread formed on an inner wall thereof screwed with the outer thread of the spout of the box body, a channel extending outwardly from an inner bottom wall thereof and located inside the spout and provided with an outer diameter smaller than an inner diameter of the spout, and a division located inside the channel and provided with a through hole at a center thereof and the at least one water box outlet;
wherein the valve pin has an end formed with a mounting slot and other end formed with a constraining block, and inserted into the spring and the through hole of the division to constrain the spring between the division and the constraining block;
wherein the sealing member has a fixing hole formed at a center thereof sleeved on the valve pin and fastened in the mounting slot of the valve, such that the sealing member covers the channel to close the channel subject to an elastic force of the spring;
wherein when the water box is assembled with the base, the body part of the box body is located in the first receiving cavity portion and contacted against the sidewall of the first receiving cavity portion, the spout of the box body and the box cover are located in the second receiving cavity portion, the pillar pushes against the valve pin to move the valve pin upwardly to compress the spring, so as to make the sealing member leave from the channel and enable space inside the water box to be in communication with the second receiving cavity portion.

6. The facial steaming apparatus according to claim 2, wherein the steam spray head has at least one fastening block, and a sidewall of the steamed object receiving cavity of the main body has at least one fastening slot engagable with the at least one fastening block.

7. The facial steaming apparatus according to claim 2, further comprising a flask detachably disposed in the steamed object receiving cavity of the main body for receiving a to-be-steamed object.

8. The facial steaming apparatus according to claim 1, wherein the adjusting knob is configured to switch among a turn-off state, a towel heating state, a fruit-vegetable steaming state and a pure-water steaming state.

9. The facial steaming apparatus according to claim 1, further comprising a UV lamp located in the steamed object receiving cavity and having a bottom thereof in contact with the bottom wall of the steamed object receiving cavity.
